(19) United States
(12) Plant Patent Application Publication
(10) Pub. No.: US 2003/0056259 P1
(43) Pub. Date: Mar. 20, 2003

(54) Chrysanthemum plant named 'Lunar Time'

(76) Inventor: Mark Roland Boeder, J H Den Haag (NL)

Correspondence Address: HOWREY SIMON ARNOLD & WHITE, LLP, BOX 34, 301 RAVENSWOOD AVE., MENLO PARK, CA, 94025, US

(21) Appl. No.: 09/957,487

(22) Filed: Sep. 19, 2001

(51) Int. Cl. A01H 5/00

(52) U.S. Cl. PLT/286

(57) ABSTRACT

A new variety of Chrysanthemum plant named ‘Lunar Time,’ having cream red purple single type flowers, a slightly domed capitulum form and an upright growth habit.

### BACKGROUND OF THE INVENTION

The present invention comprises a new and distinct cultivar of Chrysanthemum botanically known as Dendranthema grandiflora, and referred to by the cultivar name ‘Lunar Time.’

‘Lunar Time,’ identified as 20383-0 originated from a cross made by Mark Boeder, in a controlled breeding program in Chichester, West Sussex, United Kingdom. The female parent of the new variety is an undistributed, unpatented seedling identified as P452C3 and the male parent is the undistributed, unpatented seedling identified as P333C6. The new variety ‘Lunar Time’ has been asexually reproduced by vegetative cuttings at Chichester, West Sussex, United Kingdom and the distinguishing characteristics are retained through successive generations of asexual reproduction.

### BRIEF SUMMARY OF THE INVENTION

‘Lunar Time’ is a pot type of Chrysanthemum plant variety having a flat capitulum form and large cream red purple single flowers.

### Comparison with Parent

Plants of the new Chrysanthemum variety ‘Lunar Time’ are similar to the parent cultivar P452C3 in plant habitat and growth rate. In side-by-side comparisons in Chichester, West Sussex, United Kingdom, under commercial practice, plants of the new Chrysanthemum variety ‘Lunar Time’ differed from plants of the parent cultivar P452C3 in the following characteristics.

1. The new variety ‘Lunar Time’ produces large cream red purple single flowers whereas the parent cultivar P452C3 produces smaller red/yellow bi-coloured single flowers.

2. The new variety Lunar Time has a faster response time than the parent variety P452C3.

3. Plants of the new variety ‘Lunar Time’ have larger flowers than the parent variety P452C3.

### Comparison with Other Varieties

1. There are no known direct comparable varieties to Lunar Time, the variety having distinctive cream red purple coloured flowers.

### BRIEF DESCRIPTION OF ILLUSTRATIONS

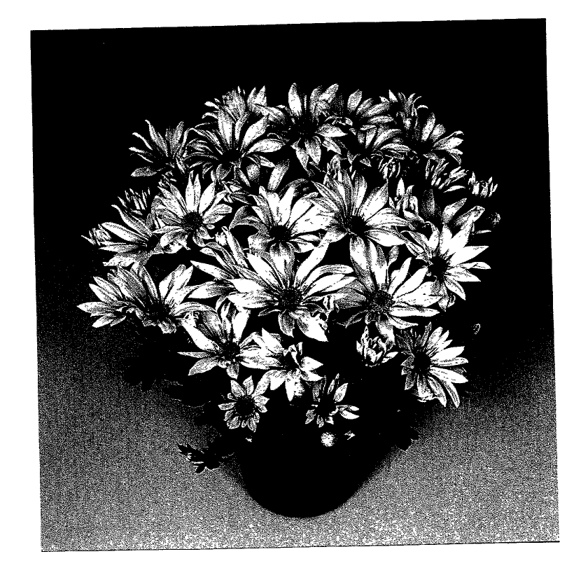

Typical specimens of the plant and flowers for the new Chrysanthemum variety ‘Lunar Time’ are shown in the accompanying photograph. The colours shown are as true as possible within the usual limits of this kind of illustration. FIG. 1 is a whole plant view of the new variety ‘Lunar Time’ grown in a pot. The plant shown in the illustration is 56 days from the start of Short Days.

### DETAILED BOTANICAL DESCRIPTION

The following description of the new Chrysanthemum variety ‘Lunar Time’ is of plants grown in a greenhouse in Chichester, West Sussex, United Kingdom in the month of June. The cultivar has not been observed under all possible environmental conditions. The phenotype may vary significantly with variations in the environment such as temperature, length of day and light intensity, without any variance in genotype. The commercial classification of the new variety is a pot Chrysanthemum.

Plants of the new variety have been grown successfully under temperature conditions averaging about 18° C. at night and about 24° C. to 25° C. during the day under light conditions of about 5,000 to 6,000 foot candles. The plants respond well to the use of growth retardant, such as three B9 treatments at about 2500 ppm., one application after 2 days, one application after 21 days, one application after 31 days from propagation. To produce a commercial product the plants may be pinched once. The typical container size for commercial growth is 14 cm. It has been observed that the shelf life of the new variety is about 21 days with a response time of about 7½ weeks. The new variety is suitable for growth in a temperature range of 16° C. to 25° C.

The new variety may be produced as a spray or disbud. The following description is with respect to a plant produced as Spray pot Chrysanthemums. In the description of this new Chrysanthemum variety, colour values have been taken from The Royal Horticultural Society Colour Chart (R.H.S.C.C.).

### Plant

Plant type: Pot

Habit: Upright and branching

Height: Medium/Tall 21 to 24 cm

Branching characteristics: Free and spreading

Length of Lateral Branches: 17 to 24 cm

Number of Breaks from Pinch: 4 to 6

Stem Colour: 147 A

Response Time: 52 days

Vigour: Medium/Tall

Shelf Life: Near 21 days

Disease: No observations

Pest: No observations

Growth retardant type and treatment: B9 85% 3 applications of 2.5 gram/litre at 2 days, 21 days and 31 days after sticking of unrooted cuttings. The plants were grown for two weeks in Long Day conditions (20 hours of light) and then transferred to Short Day conditions (13 hours of dark).

Propagation:

Type.—Vegetative propagation via stem cuttings.

Time to rooting.—12-14 days with soil temperatures of 18° C.

Rooting habit.—Fine and fibrous

### Foliage

Number of Leaves per Lateral Branch: 8 to 12

Compound or single: Single

Arrangement of Leaves: Alternate

Shape of leaf.—Typically 5 lobed, ovate.

Size of leaf.—Width (cm): Near 4-6 cm Length (cm): Near 6-10½ cm.

Leaf apex.—Acute.

Base.—Obtuse.

Attachment.—Petioled.

Aspect.—Slightly undulating.

Margin.—Palmately lobed,

Surface characteristics.—Top: Slightly pubescent Bottom: Slightly pubescent

Petiole: Colour: Near 137 C Length: Near 2½ cm

Venation: Net, prominent mid and lobe veins at under side Colour. upper side: Near 144 B under side: Near 144 B

Colour: Mature Leaf, upper side: Near 137 A; under side: Near 137 D Young Leaf, upper side: Near 137 A; under side: Near 137 C

### Flower

Flower type: Single (Daisy)

Flower formn: Slightly cupped with slightly reflexing petals

Flower shape: Circular

Number of blossoms per branch: 3 to 6

Inflorescence form: Cylindrical

Depth of fully expanded blossoms.—Near 2½ cm.

Diameter of fully expanded blossoms.—7 to 10 cm.

Sepals:

Number.—17 to 20.

Colour.—Near 137 B.

Length.—0.5-1.2 cm.

Width.—0.2-0.4 cm.

Texture/appearance.—Pubescent.

Pedicel:

Length.—5 to 8 cm.

Colour.—Near 137 B.

Surface.—Pubescent.

Habit.—Slightly undulating.

Strength.—Medium.

Ray Florets:

Form/shape.—Reflexing.

Texture/appearance.—Matte.

Number per flower.—20 to 25.

Length.—3½ to 5 cm; Width: 0.8 to 1½ cm.

Apex.—Pointed.

Base.—Tapered.

Margin.—Entire.

Disc florets:

Form/shape.—Cylindrical.

Texture/appearance.—Shiny.

Number per flower.—170 to 250.

Length.—Near 0.5 to 0.7 cm; Width <0.1 cm.

Diameter of disc.—1.2 to 2 cm.

Fragrance: Faint

Flower Bud (at onset of colour):

Length.—Near 1.0 cm.

Diameter.—Near 1.4 cm.

Form/shape.—Globular.

General flower colour:

1. Ray florets, upper side.—Immature: Near 10 B with flecking of 54 B to base Mature: Near 10 C with flecking of 54 B to base Older/Fading: Near 10 C with flecking of 54 B to base.

2. Ray florets, under side.—Immature: Near 10 B with flecking of 54 A over lower half of petal Mature: Near 10 B with flecking of 54 B over most of petal Older/Fading: Near 10 C with flecking of 54 B over most of petal.

3. Disc florets.—Immature: Near 151 B Mature: Near 6 A Older/Fading: Near 6 A.

4. Bud.—Near 138 B

Flower progression: Reflexing of petals increases slightly with age.

Other Distinguishing Characteristics: Unique bi-colour effect to flower

### Reproductive Organs

Ray Florets: Pistillate

Stigma colour.—Near 6 C.

Stigma shape.—Forked.

Style colour.—Near 6 C.

Style length.—Near 0.5 cm.

Disc Florets: Pistillate

Stigma colour.—Near 6 C.

Stigma shape.—Forked.

Style colour.—Near 6 C.

Style length.—Near 0.1 to 0.2 cm.

Staminate.—

Stamen number.—Near 25

Anther shape.—Cylindrical.

Anther colour.—Near 6 A.

Pollen colour.—Near 14 A.

Pollen quantity.—Little.

What is claimed is:

1. A new and distinct variety of Chrysanthemum plant, substantially as described and illustrated herein.
